(19) United States
(12) Patent Application Publication
(10) Pub. No.: US 2019/0000415 A1
(43) Pub. Date: Jan. 3, 2019

(54) ULTRASOUND SYSTEM AND METHOD FOR ACQUISITION PARAMETER DETERMINATION

(71) Applicants: CARESTREAM HEALTH, INC., Rochester, NY (US); University of Rochester, Rochester, NY (US)

(72) Inventors: Ajay ANAND, Rochester, NY (US); Michael S. RICHARDS, Rochester, NY (US)

(21) Appl. No.: 16/019,858

(22) Filed: Jun. 27, 2018

Related U.S. Application Data
(60) Provisional application No. 62/598,494, filed on Dec. 14, 2017.
(60) Provisional application No. 62/525,777, filed on Jun. 28, 2017.

(51) Int. Cl. A61B 8/08; G01S 15/89; G01S 7/52; A61B 8/00

(52) U.S. Cl. CPC A61B 8/0891; G01S 15/8984; A61B 8/461; G01S 7/52085; G01S 7/5202

(57) ABSTRACT

A method for ultrasound imaging acquires an initial set of ultrasound images of a region of interest at an initial time t0 using a pulsed wave velocity imaging mode and one or more subsequent sets of ultrasound images in pulsed wave velocity imaging mode over the same region at a predetermined frame rate. The relative displacement between acquired frames is computed. A pulse wave velocity distribution is generated according to the computed displacement by comparing arrival times of the pulse wave at different locations over the region. A subset of pulse wave velocity values from the distribution is identified according to standard deviation or other statistical criterion. Statistical results are computed according to the subset of pulse wave velocity values. Computed statistical results are displayed.

### CROSS REFERENCE TO RELATED APPLICATIONS

This application claims the benefit of U.S. Provisional application U.S. Ser. No. 62/598,494 provisionally filed on Dec. 14, 2017, entitled “ULTRASOUND SYSTEM AND METHOD FOR ACQUISITION PARAMETER DETERMINATION”, in the names of Ajay Anand and Michael Richards, incorporated herein by reference in its entirety.

This application claims the benefit of U.S. Provisional application U.S. Ser. No. 62/525,777 provisionally filed on Jun. 28, 2017, entitled “ULTRASOUND SYSTEM AND METHOD FOR ACQUISITION PARAMETER DETERMINATION”, in the names of Ajay Anand and Michael Richards, incorporated herein by reference in its entirety.

### SPONSORED RESEARCH AND DEVELOPMENT

This research was funded in part by CEIS (Center for Emerging & Innovative Sciences), an Empire State Development-designated Center for Advanced Technology.

### TECHNICAL FIELD

The disclosure relates generally to the field of ultrasound systems and methods and more particularly to apparatus and methods that determine optimal acquisition parameters. More specifically, the disclosure relates to determining optimal acquisition parameters for ultrasound pulse wave imaging.

### BACKGROUND

Ultrasound imaging systems/methods are known medical devices, such as those described, for example, in U.S. Pat. No. 6,705,995 (Poland), U.S. Pat. No. 5,370,120 (Oppelt), US 2015/0141821 (Yoshikawa), and U.S. Pat. No. 8,285,357 (Gardner), all of which are incorporated herein in their entirety. Various applications for diagnostic ultrasound systems are given, for example, in the article entitled “Ultrasound Transducer Selection In Clinical Imaging Practice”, by Szabo and Lewin, Journal of Ultrasound Medicine, 2013; 32:573-582, incorporated herein by reference in its entirety.

Ultrasound utilizes sound waves at frequencies higher than those perceptible to the human ear. Ultrasonic images known as sonograms are generated as a result of pulsed ultrasonic energy that has been directed into tissue using a probe. The probe obtains echoed sound energy from the internal tissue and provides signal content that represents the different sound reflectivity exhibited by different tissue types. This signal content is then used to form images that visualize features of the internal tissue. Medical ultrasound, also known as diagnostic sonography or ultrasonography, is used as a diagnostic imaging technique used to help visualize features and operation of tendons, muscles, joints, vessels and internal organs of a patient.

A number of advanced diagnostic tools have been demonstrated, based on continuing developments in ultrasound emission and detection, along with ongoing improvements in computation speed and in image processing and presentation techniques.

Of particular interest for cardiovascular applications, for example, is the development and use of ultrasound signals for noninvasive characterization of arterial stiffness within local anatomical regions. This topic has been widely discussed in the modern biomedical ultrasound literature.

Background information of particular interest can be found in the following references: (1) Jordi Calabia et. al. “Doppler ultrasound in the measurement of pulse wave velocity: agreement with the Complior method” published in Cardiovascular Ultrasound, 201, 9; 13; and (2) Jonathan Vappou et al. “Aortic Pulse Wave Velocity Measured By Pulse Wave Imaging (Pwi): A Comparison With Applanation Tonometry” Artery Research, 2011 Jun. 1; 5(2):pp. 65-71.

It has been found that large arteries are not simple tube conduction structures. These arteries help to moderate systolic pressure increases and maintain sufficient diastolic level to guarantee myocardial perfusion. With the identification of new diseases and risk factors, it has been seen that these arteries lose their natural elasticity leading to high systolic and low diastolic blood pressure levels, which determine high pulse pressure.

Arterial stiffness is being recognized as a useful biomarker for evaluating cardiovascular risk and for detection of incipient vascular disease. Recent research suggests that this parameter can serve as an independent predictor of cardiovascular mortality among elderly, hypertensive, and diabetic patients, among patients having chronic renal failure, and in the general population. Guidelines of the European Societies of Hypertension and Cardiology (2007-2009) postulate arterial stiffness assessment, measurement of the carotid plaque and ankle/brachial index as markers of vascular status. Any change in measured values may define a state of vasculopathy that significantly increases patient risk.

Among methods for evaluating arterial stiffness, the most widely used metric in the literature is aortic pulse wave velocity (PWV). PWV relates to the speed at which the pulse wave travels on an arterial segment. PWV is often measured over the region that extends from the aortic arch or common carotid artery to the common femoral artery. PWV can be measured in a number of ways. In some methods, PWV is estimated as the ratio between change in flow (Q) and change in cross-sectional area (A) (PWV=dQ/dA) during the reflection-free period of the cardiac cycle. Conventionally, the pulse wave velocity measurement itself has been obtained using pressure transducers or employing arterial tonometry.

Ultrasound signals have been used for PWV evaluation using carotid-femoral PWV measurements. Pulsed Doppler ultrasound measurements, using a linear array probe, are timed with ECG (electrocardiogram) or other biometric signals at the carotid artery and at the femoral artery. The pulse wave transit time is computed and averaged to obtain measured data. Pulse Wave Imaging (PWI) using ultrasound is well suited for localized measurements and is generally more sensitive to changes in local pathology than are more global measurement modes. For PWI, ultrasound signals are acquired at high frame rates, with arterial wall displacement computed using cross-correlation methods.

PWV imaging, however, can be particularly sensitive to various acquisition parameters used by the operator, including frame rate. The optimal frame sampling rate for one patient or for one anatomical region may not be well suited for use with a different patient or over a different anatomical region, even for the same patient. This is because the optimal settings depend on the propagation velocity of the pulse wave that, in turn, depends on the local vessel wall properties, disease state of the blood vessel, and the overall hemodynamic status of the patient. The sonographer or other operator may have little experience or guidance in selecting the appropriate frame rate or related operational parameter for a PWV exam. Thus, it can be appreciated that methods for sensing and determining the appropriate frame rate and other suitable parameter data can be of particular value for effective delivery and measurement of PWV signals.

### SUMMARY

An object of the present disclosure is to advance the art of ultrasound imaging and overall ultrasound system operation, directed more particularly to pulsed wave imaging.

These objects are given only by way of illustrative example, and such objects may be exemplary of one or more embodiments of the invention. Other desirable objectives and advantages inherently achieved may be apparent to those skilled in the art. The invention is defined by the appended claims.

According to one aspect of the disclosure, there is provided a method for ultrasound imaging comprising: a) acquiring an initial set of ultrasound images of a region of interest at an initial time t0 using a pulsed wave velocity imaging mode; b) acquiring one or more subsequent sets of ultrasound images in pulsed wave velocity imaging mode over the same region at a predetermined frame rate; c) computing a relative vessel wall displacement induced by pulse waves between frames acquired in a) and b); d) generating a pulse wave velocity distribution according to the computed displacement by comparing arrival times of the pulse wave at two different locations within the region of interest; e) identifying a subset of pulse wave velocity values from the distribution according to standard deviation or other statistical criterion; f) computing statistical results according to the subset of pulse wave velocity values; and g) displaying the computed statistical results on a display.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIGS. 1A and 1B show exemplary ultrasound systems.

FIG. 2 shows a schematic of an exemplary ultrasound system.

FIG. 3 illustrates a Sonographer using an exemplary ultrasound system.

FIG. 4 is a schematic diagram showing wave signals for carotid-femoral measurement.

FIG. 5 is a logic flow diagram showing steps for frame rate determination.

### DETAILED DESCRIPTION OF THE EMBODIMENTS

The following is a detailed description of the preferred embodiments, reference being made to the drawings in which the same reference numerals identify the same elements of structure in each of the several figures.

Where they are used in the present disclosure, the terms “first”, “second”, “third”, and so on, do not necessarily denote any ordinal or priority relation, but are simply used to more clearly distinguish one element from another.

Medical ultrasound (also known as diagnostic sonography or ultrasonography) is a diagnostic imaging technique based on the application of ultrasound, used to display internal body structures such as tendons, muscles, joints, vessels and internal organs.

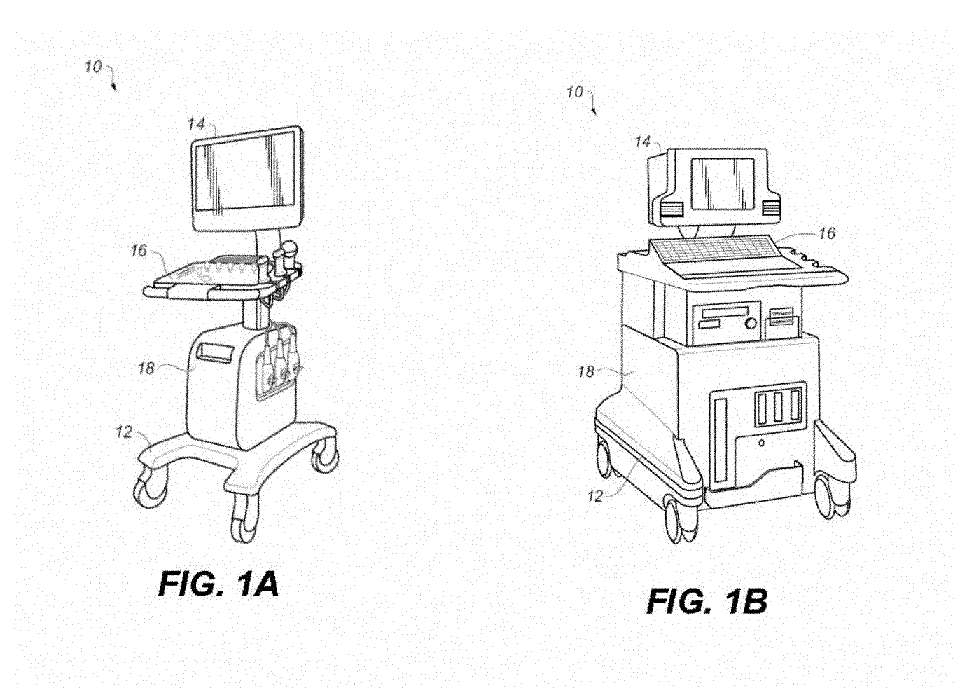

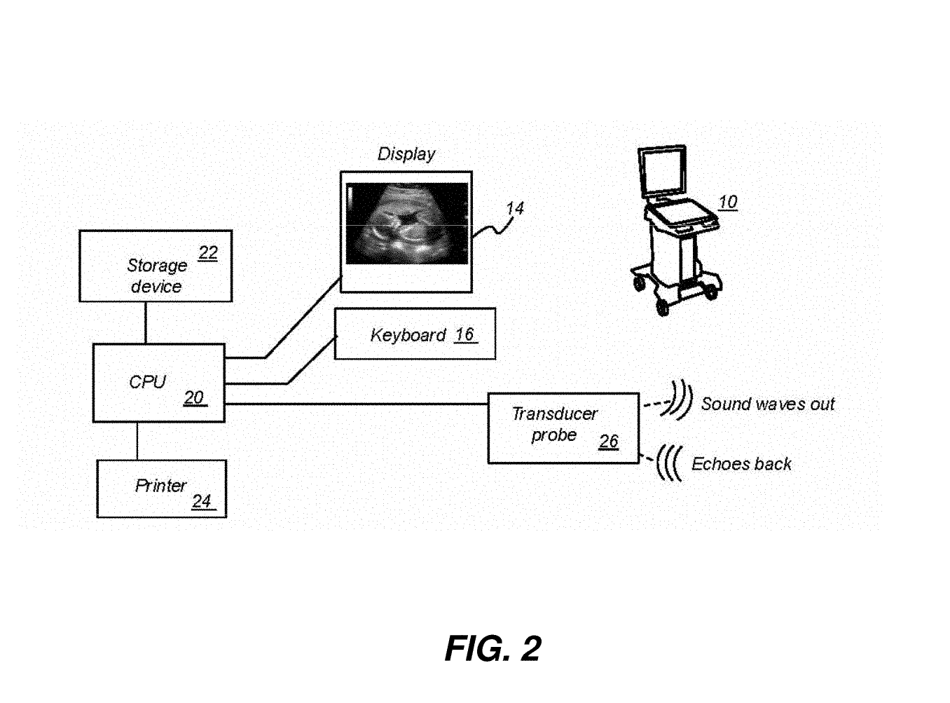

FIGS. 1A-1B and FIGS. 2-3 show exemplary portable ultrasound systems 10 that use a cart/base/support, cart 12, a display/monitor 14, one or more input interface devices 16 (such as keyboard or mouse), and a generator 18. The display/monitor 14 can also be a touchscreen to function as an input device. As illustrated, the ultrasound system 10 can be a mobile or portable system designed to be wheeled from one location to another. As FIG. 2 shows, the ultrasound system 10 has a central processing unit CPU 20 that provides control signals and processing capabilities. CPU 20 is in signal communication with display 14 and interface device 16, as well as with a storage device 22 and an optional printer 24. A transducer probe 26 provides the ultrasound acoustic signal and generates an electronic feedback signal indicative of tissue characteristics from the echoed sound.

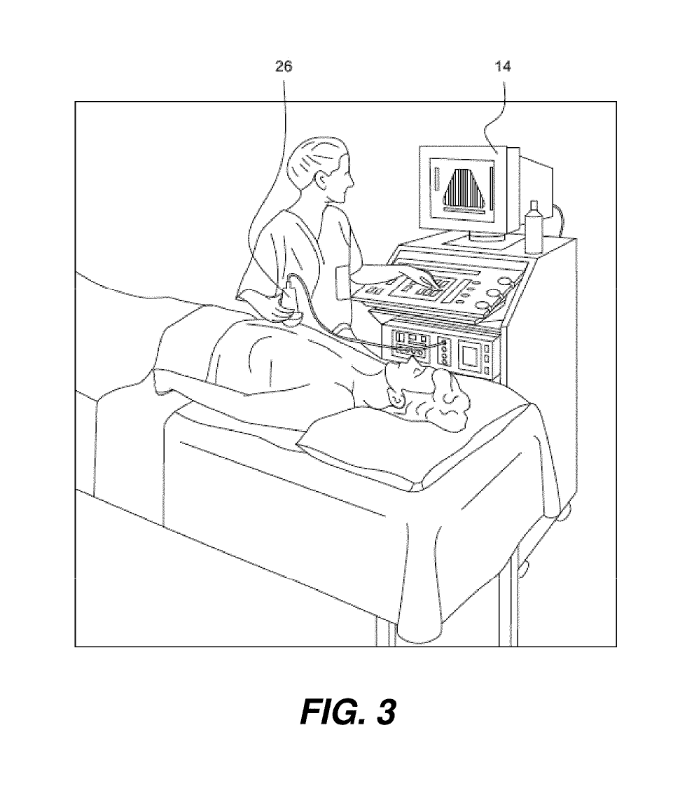

FIG. 3 shows an example of an ultrasound system 10 in use with an image provided on display/monitor 14.

The ultrasound system, shown by way of example in FIGS. 1A and 1B, can include an image processing system, a user interface and a display. The image processing system includes a memory and a processor. Additional, different or fewer components may be provided in the system or image processing system. In one embodiment, the system is a medical diagnostic ultrasound imaging system. The memory is a RAM, ROM, hard drive, removable media, compact disc, DVD, floppy disc, tape, cache memory, buffer, capacitor, combinations thereof or any other now known or later developed analog or digital device for storing information. The memory is operable to store data identifying a selected point for identifying a region of interest. The memory is operable to store data identifying one or a plurality of region of interest. Information from the user interface indicating a position on an image on the display is used to determine a spatial relationship of a user selected point to a scanned region or image position. The selected point is an individual or single point in one embodiment that may be a point selected within a line, area or volume. Additional or different information may be also stored within the memory. The processor is general processor, application specific integrated circuit, digital signal processor, controller, field programmable gate array, digital device, analog device, transistors, combinations thereof or other now known or later developed devices for receiving analog or digital data and outputting altered or calculated data. The user input is a track ball, mouse, joy stick, touch pad, buttons, slider, knobs, position sensor, combinations thereof or other now known or later developed input devices. The user input is operable to receive a selected point from a user. For example, the user positions a cursor on an image displayed on the display. The user then selects a position of the cursor as indicating a point for a region of interest. The display is a CRT, LCD, plasma screen, projector, combinations thereof or other now known or later developed devices for displaying an image, a region of interest, region of interest information and/or user input information.

Different types of images can be formed using sonographic instruments. The most well-known type is a B-mode image, which displays the acoustic impedance of a two-dimensional cross-section of tissue. Other types of image can display blood flow, motion of tissue over time, the location of blood, the presence of specific materials, the stiffness of tissue, or the anatomy of a three-dimensional region.

Accordingly, the system of FIGS. 1A-3 are typically configured to operate within at least two different ultrasound modes. As such, the system provides means to switch between the at least two different ultrasound modes. Such a two-mode configuration and means for switching between modes are well known within the ultrasound technology.

Clinical modes of ultrasound used in medical imaging include the following:
A-mode: A-mode (amplitude mode) is the simplest type of ultrasound. A single transducer scans a line through the body with the echoes plotted on screen as a function of depth. Therapeutic ultrasound aimed at a specific tumor or calculus also uses A-mode emission to allow for pinpoint accurate focus of the destructive wave energy.
B-mode or 2D mode: In B-mode (brightness mode) ultrasound, a linear array of transducers simultaneously scans a plane through the body that can be viewed as a two-dimensional image on screen. Sometimes referred to as 2D mode, this mode is effective for showing positional and dimensional characteristics of internal structures and is generally the starting point for exam types that use other modes.
C-mode: A C-mode image is formed in a plane normal to a B-mode image. A gate that selects data from a specific depth from an A-mode line is used; the transducer is moved in the 2D plane to sample the entire region at this fixed depth. When the transducer traverses the area in a spiral, an area of 100 cm² can be scanned in around 10 seconds.
M-mode: In M-mode (motion mode) ultrasound, pulses are emitted in quick succession. With each pulse, either an A-mode or B-mode image is acquired. Over time, M-mode imaging is analogous to recording a video in ultrasound. As the organ boundaries that produce reflections move relative to the probe, this mode can be used to determine the velocity of specific organ structures.
Doppler mode: This mode makes use of the Doppler effect in measuring and visualizing blood flow.
Color Doppler: Velocity information is presented as a color-coded overlay on top of a B-mode image. This mode is sometimes referred to as Color Flow or color mode.
Continuous Doppler: Doppler information is sampled along a line through the body, and all velocities detected at each point in time are presented (on a time line).
Pulsed wave (PW) Doppler: Doppler information is sampled from only a small sample volume (defined in 2D image), and presented on a timeline.
Duplex: a common name for the simultaneous presentation of 2D and (usually) PW Doppler information. (Using modern ultrasound machines, color Doppler is almost always also used; hence the alternative name Triplex.).
Pulse inversion mode: In this mode, two successive pulses with opposite sign are emitted and then subtracted from each other. This implies that any linearly responding constituent will disappear while gases with non-linear compressibility stand out. Pulse inversion may also be used in a similar manner as in Harmonic mode.
Harmonic mode: In this mode a deep penetrating fundamental frequency is emitted into the body and a harmonic overtone is detected. With this method, noise and artifacts due to reverberation and aberration are greatly reduced. Some also believe that penetration depth can be gained with improved lateral resolution; however, this is not well documented.

A sonographer, ultrasonographer, clinician, practitioner, or other clinical user, is a healthcare professional (often a radiographer but may be any healthcare professional with the appropriate training) who specializes in the use of ultrasonic imaging devices to produce diagnostic images, scans, videos, or 3D volumes of anatomy and diagnostic data.

As noted in the background material given earlier, measurement of the aortic pulse wave velocity (PWV) is regarded as very useful in the region extending from the aortic arch or common carotid artery to the common femoral artery.

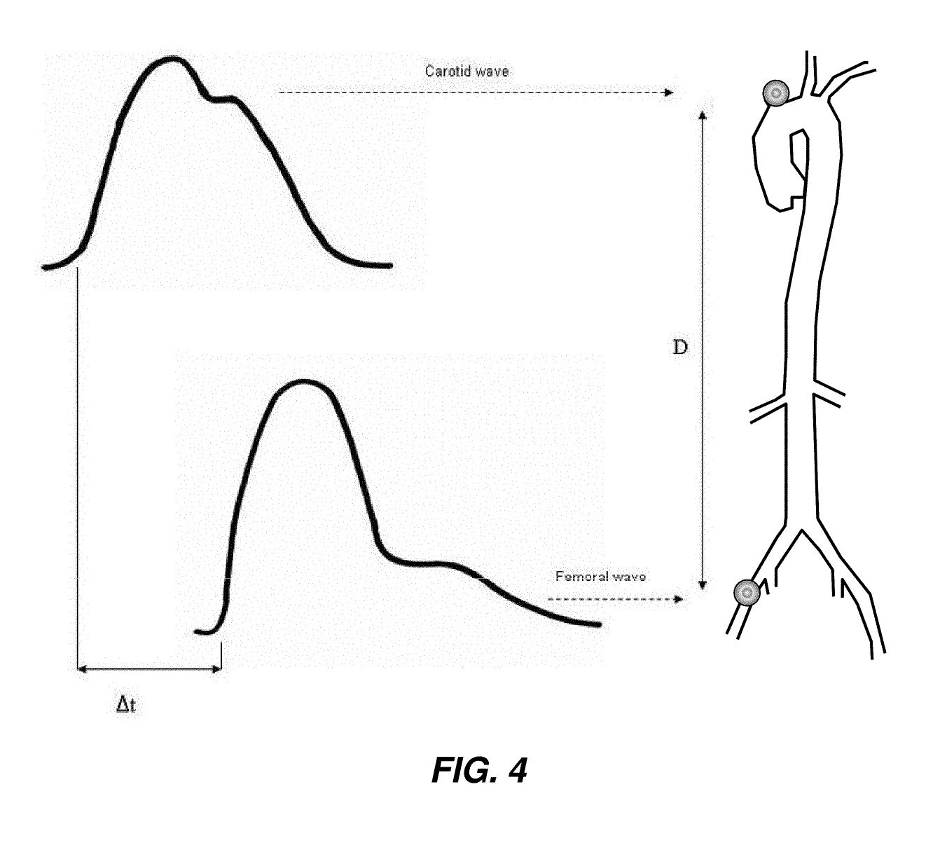

As shown schematically in FIG. 4, measurement of carotid-femoral PWV is made by dividing the distance D from the carotid point to the femoral point by the so-called transit time Δt that gives the time of travel of the pulse wave over the distance:

PWV=D(meters)/Δt(seconds).

The transit time measures the time of travel of the foot of the wave over a known distance. Transit time Δt is estimated by the foot-to-foot method. The foot of the wave is defined at the end of diastole, where the steep rise of the waveform begins.

Measurement methods have been shown to be highly reliable, but present disadvantages. Measurement requires specific devices and software and may not always be accurate in practice, due to difficulties in reliably recording pulse wave signals. Furthermore, the time required for the exploration is not negligible.

In practice, one of the limitations of conventional test methods is that a global PWV estimate is provided. As invasive testing has shown, PWV varies significantly along the entire arterial tree due to variations in both the geometry and structure of the arteries. Therefore, the carotid-to-femoral PWV estimate represents an average of the local PWV values all along this arterial segment. Different arteries tend to stiffen at different rates and magnitudes during aging, with central arteries undergoing significantly higher stiffening than peripheral arteries. More importantly, a large number of pathologies result in localized arterial stiffening. For these reasons, it can be of particular value to measure PWV locally.

A number of imaging methods have been proposed for the purpose of estimating PWV locally, primarily using MRI (magnetic-resonance imaging) and ultrasound. Pulse Wave Imaging (PWI) using ultrasound has been previously reported in the literature for both qualitative visualization of the pulse wave propagation in real time and for quantitative estimation of the PWV within the imaged arterial segment. PWI relies on tracking the arterial wall displacement using cross-correlation methods on RF ultrasound signals acquired at high frame rates. Measurements of Pulse Wave Velocity (PWV) have been found particularly useful for this purpose, allowing more precise information on circulatory condition over various regions of the anatomy to be obtained.

Noninvasive quantification of regional arterial stiffness, such as can be derived from measurement of the Pulse Wave Velocity (PWV), has been considered to be of considerable clinical value. Pulse waves are flow velocity, pressure, and diameter waves generated at the ejection phase of the left ventricle. In an idealized model, the propagation speed of the wave (i.e., the PWV) is directly related to the Young's modulus of the artery or other blood vessel. Young's modulus, also known as the elastic modulus, provides a measure of material stiffness.

Ultrasound-based techniques have been developed to measure the Pulse Wave Velocity for its diagnostic value. The ultrasound-based measurement provides a local or regional estimate that is viewed by some practitioners to be more readily correlated with spatial positions on an artery compared to global estimates conventionally obtained with non-imaging techniques. The ultrasound-based approach can be further extended to image the pulse wave such that the propagation patterns of the pulse wave can be visualized in addition to obtaining a single PWV value. Pulse Wave Imaging (PWI) has been investigated as a tool for characterizing the propagation of the pulse wave along the aorta and estimating the regional PWV. Related clinical applications include detection of abdominal aortic aneurysms and subsequent follow up.

The ultrasound approach is based on tracking micron-sized displacements that are induced by the propagating pulse wave as it moves along the vessel wall. The displacement estimation approach is sometimes referred to as speckle tracking, and can be implemented by tracking the echo shifts in the radio frequency signal.

A variety of methods have been proposed in the literature that are based on classic time-delay estimation, and phase tracking using autocorrelation-based estimators. The displacement estimation is performed at each of several lateral locations along the vessel wall over the extent of the lateral transducer width. Depending on the exact algorithm that is used, several adjacent lateral locations could be included in estimating a single PWV estimate. At each lateral vessel wall location, the temporal displacement profile has a sharp rise to peak displacement, followed by a transient decay back to baseline. One approach is to record the time instant when the sharp displacement rise (“knee of the waveform”) occurs and thus create a regression plot of arrival time as a function of lateral distance. The slope of the curve then provides an indicator of pulse wave velocity. The greater the number of scanlines (i.e. lateral locations used in the slope estimation), the lower the expected variance in the calculated velocity estimate.

Although algorithms that perform the displacement estimation are straightforward, the accuracy of the resulting estimates depends on a variety of acquisition parameters. One parameter is the acquisition frame rate. Since the pulse wave displacement waveform has sharp transient characteristics (i.e. rapid rise and fall times) and since the entire pulse wave traverses a single location within tens or few hundreds of milliseconds, the waveforms need to be sufficiently well-sampled in order to be able to detect the features of interest (such as the “knee” of the waveform, described previously).

Conversely, if the frame rate is very high, the inter-frame displacement is small. This, in turn, affects the accuracy of the displacement estimates because of challenges in tracking very small displacements that can be close to the noise floor. Judicious choice of the inter-frame time interval is thus desired, considered important for promoting reliable tracking of the displacements without introducing jitter, bias, and other undesirable tracking errors. Moreover, in a diseased artery, since the pulse wave velocity increases due to stiffening and the waveform shape changes as well, it may not be possible to characterize a priori the optimal frame rate for use in particular clinical situations.

Applicants have recognized, from recent ex vivo data acquired by Applicants during the course of research, the variability of the pulse wave velocity estimates with frame rate (Frames Per Second, or FPS), as illustrated in Table 1 below. Applicants' data was acquired in a flow phantom setup with an aorta model fabricated using 10% PVA polymer (Polyvinyl alcohol).

TABLE 1

| Frame Rate (FPS) | PWV (m/s) | E (kPa) |
|---|---|---|
| 557 | 4.53 | 145.9 |
| 1055 | 2.79 | 55.2 |
| 1908 | 2.72 | 52.6 |

In a clinical setting, this relative level of variability in measurement can be highly troubling, raising concerns about data validity and causing some confusion about which value may be correct.

Applicants' method addresses this problem by proposing a workflow approach that improves confidence in determining the correct PWV value or range, and guides the user to the optimal set of acquisition parameters. For example, in one configuration, the Applicants' method is integrated into software developed to implement Pulse Wave Imaging on a medical ultrasound system.

Applicants' method determines the optimal frame rate or range of frame rates which provides the preferred/desired/accurate value for the Pulse Wave Velocity in the clinical situation being imaged.

### Sequence for Frame Rate Determination

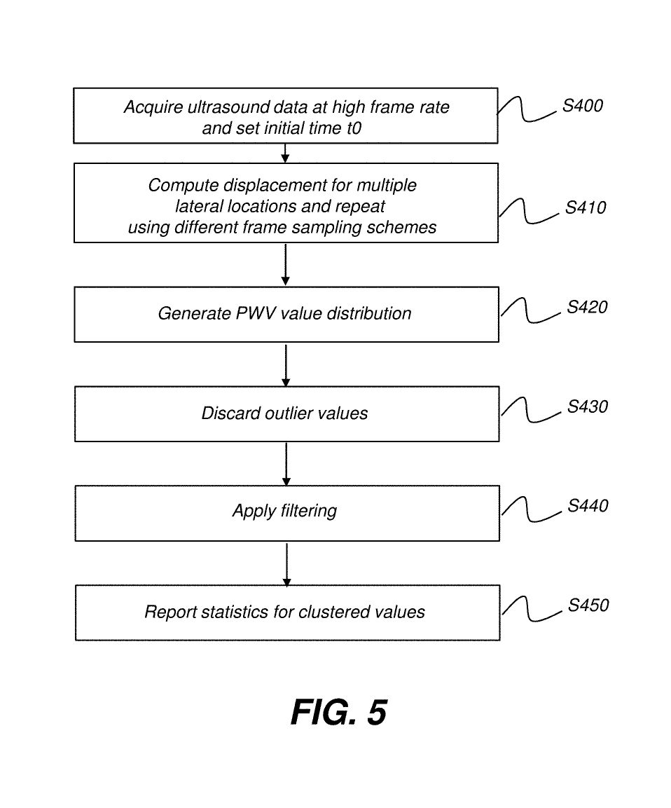

Applicants' method can be implemented using one or more of the steps described following and shown in summary in the logic flow diagram of FIG. 5.

In an acquisition step S400, acquire ultrasound echo data at the highest allowable frame rate. Set an initial time (for example, t0) to represent an instant prior to the onset of the pulse wave at a particular location.

In a computation step S410, compute the displacement (or velocity) at each spatial lateral location using two or more different displacement calculation schemes, for example:

(a) Frame-to-frame, in a sampling pattern that does not skip frames from adjacent acquisitions. This allows computation of the incremental relative displacement between adjacent frames and accumulates a running total that is indicative of the total displacement at every frame time.

(b) Frame-to-frame, with frame skipping. The frame-to-frame displacement is computed but with sampling at different time intervals, skipping frames in a sequence that effectively increases the magnitude of the displacement computed in every pair of frames compared. As in scheme (a), total displacement is estimated by cumulatively adding displacements from each pair of frames compared; however, in scheme (b) the sampling pattern is different. Scheme (b) can be repeated by systematically skipping a different number of frames in each iteration, up to a predefined maximum.

(c) Reference-frame based acquisition. Here, the total displacement is calculated by comparing each sampled frame with respect to frame 1 that is nominally acquired at initial time t0 and that represents the initial position.

Computation step S410 can optionally be repeated for some portion or all of the lateral locations.

In an initial distribution generation step S420, a PWV value is computed using each of the approaches applied in step S410 (step 2, above). A distribution of the candidate PWV values is then obtained as an initial set of PWV values.

A discard step S430 can effectively discard outlier values from the distribution in order to generate a subset of pulse wave velocity values. In practice, it has been found that a subset of the PWV values cluster together with minimal variability, while outlier values resulting from sub-optimal frame comparison choices are nominally separated from the cluster. These outlier values can be removed from subsequent computation and analysis. Statistical measurements can be used to identify a subset of pulse wave velocity values usable for subsequent computation, such as values within a predetermined standard deviation (SD) of a mean value, for example.

In an optional filtering step S440, other metrics such as using cross-correlation coefficients (computed during the displacement estimation for each of the approaches in Step 2) can be used as a filter to further reduce the subset of candidate values from the clustered subset obtained in Step 4 above.

In a reporting step S450, following outlier removal from earlier processing, a range from the cluster of PWV values, such as a mean value +/−standard deviation, or other descriptive statistic that is representative of the cluster (including median, mean, and other statistics) can be reported, displayed to the operator or stored for future processing.

Various frame selection schemes for frame sampling, described above with relation to the FIG. 5 sequence, have been reported in the literature. Applicants' method provides a method wherein any of a number of schemes can be applied to the same data set (the same acquired set of frames). Using this processing, a cluster of measurement values can be detected and the optimal value can be selected using known descriptive statistical approaches.

According to an embodiment, the calculated pulse wave velocity distribution can be used to automate selection of a suitable frame rate for subsequent PWV imaging for a particular location.

Applicants' method can be configured/arranged as a computer medium or software package integrated with the main system software for pulse wave imaging on a medical ultrasound system. The approach can also be applied to other ultrasound imaging modes (such as strain elastography, vessel wall distension measurements, and the like) where displacements are estimated from ultrasound echo data and optimal range of frame rates for data sampling is significant.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A method for ultrasound imaging, comprising:
a) accessing an initial set of ultrasound images of a region of interest at an initial time t0 acquired using a pulsed wave velocity imaging mode;
b) accessing at least one subsequent set of ultrasound images of the region of interest acquired using a pulsed wave velocity imaging mode at a predetermined frame rate;
c) computing a relative vessel wall displacement induced by pulse waves between frames acquired in steps a) and b);
d) generating a pulse wave velocity distribution according to the computed relative vessel wall displacement by comparing arrival times of the pulse wave at two different locations within the region of interest;
e) identifying a subset of pulse wave velocity values of the generated pulse wave velocity distribution according to a statistical criterion;
f) computing at least one statistical result according to the identified subset of pulse wave velocity values; and
g) displaying, on a display, the computed at least one statistical result.

2. The method of claim 1 further comprising discarding outlier values from the pulse wave velocity distribution prior to identifying the subset of pulse wave velocity values.

3. The method of claim 1 further comprising filtering and discarding outlier values from the subset of pulse wave velocity values prior to computing the at least one statistical result.

4. A computer storage medium having instructions stored therein for causing a computer to perform the method of claim 1.

5. A computer product embodied in a computer readable medium for performing the steps of claim 1.

6. A computer storage product having at least one computer storage medium having instructions stored therein causing one or more computers to perform the method of claim 1.

7. A method, comprising:
acquiring, at a predetermined acquisition frame rate, a first set and a second set of ultrasound echo data, each acquired set of ultrasound echo data acquired at a corresponding spatial lateral location of a blood vessel unique to the set;
generating a distribution of pulse wave velocity estimates according to the first and a second sets of the ultrasound echo data;
identifying a set of pulse wave velocity values from the generated pulse wave velocity distribution according to a statistical criterion;
computing at least one statistical result according to the identified set of pulse wave velocity values; and
reporting the at least one statistical result.

8. The method of claim 7, wherein generating the distribution of pulse wave velocity estimates is accomplished by:
computing the pulse width velocity of the acquired frames; and
comparing arrival times of the pulse wave at the corresponding first spatial lateral location to arrival times at the second spatial lateral location along the blood vessel.

9. The method of claim 7 wherein reporting the at least one statistical result further comprises storing, transmitting, or displaying on a display, the at least one statistical result.

10. The method of claim 7 wherein acquiring the first and second set of ultrasound echo data comprises acquiring the data using an acquisition system's highest allowable frame rate.

11. The method of claim 7 further comprising setting an initial time to t0 to represent an instant prior to the onset of the pulse wave at an initial spatial lateral location.

12. The method of claim 7 further comprising acquiring a third set of ultrasound echo data at a second acquisition frame rate that differs from the predetermined acquisition frame rate.

13. The method of claim 7 further comprising discarding outlier values from the pulse wave velocity estimates prior to identifying the set of pulse wave velocity values.

14. The method of claim 7 further comprising filtering and discarding outlier values from the set of pulse wave velocity values prior to computing the at least one statistical result.
